(19) United States
(12) Patent Application Publication
(10) Pub. No.: US 2023/0091002 A1
(43) Pub. Date: Mar. 23, 2023

(54) DATA MANAGEMENT SYSTEM, DATA MANAGEMENT METHOD, AND COMPUTER PROGRAM PRODUCT

(71) Applicant: Cytoronix Inc., Kawasaki-shi (JP)

(72) Inventors: Kaita IMAI, Kanagawa (JP); Shouhei KOUSAI, Kanagawa (JP)

(73) Assignee: Cytoronix Inc., Kawasaki-shi (JP)

(21) Appl. No.: 18/060,271

(22) Filed: Nov. 30, 2022

Related U.S. Application Data
(63) Continuation of application No. PCT/JP2020/028934, filed on Jul. 28, 2020.

(51) Int. Cl. C12M 3/00; C12M 1/34

(52) U.S. Cl. CPC C12M 3/00; C12M 41/30

(57) ABSTRACT

A data management system according to an embodiment includes a first storage control unit, a second storage control unit, and a third storage control unit. The first storage control unit controls storing, in a memory, one or more culture conditions indicating conditions for culturing a cell. The second storage control unit controls storing, in the memory, one or more pieces of measurement data for the cell cultured based on any one of the culture conditions. The third storage control unit controls storing, in the memory, one or more pieces of the measurement data designated among the pieces of the measurement data and one or more of the culture conditions designated among the culture conditions, in association with each other.

### CROSS-REFERENCE TO RELATED APPLICATIONS

This application is a continuation of International Application No. PCT/JP2020/028934, filed on Jul. 28, 2020, the entire contents of which are incorporated herein by reference.

### FIELD

Embodiments described herein relate to a data management system, a data management method, and a computer program product.

### BACKGROUND

A technique has been proposed for which cultured cells are measured using a measurement device such as a microscope to obtain measurement data such as image data of the cells. The use of the obtained measurement data enables the state or the like of the cultured cells to be analyzed.

However, there are cases where, for example, measurement data cannot be efficiently analyzed in order to obtain an improvement point in the culture conditions of cells.

### BRIEF DESCRIPTION OF THE DRAWINGS

FIG. 1 is a diagram illustrating a configuration example of a data management system according to an embodiment;

FIG. 2 is a block diagram of a data management device according to a first embodiment;

FIG. 3 is a diagram illustrating an example of a data structure of measurement data;

FIG. 4 is a diagram illustrating an example of a data structure of culture conditions (containers);

FIG. 5 is a diagram illustrating an example of passage;

FIG. 6 is a diagram illustrating an example of grouping of the culture conditions;

FIG. 7 is a block diagram of a terminal device according to the first embodiment;

FIG. 8 is a flowchart of condition storage processing in the first embodiment;

FIG. 9 is a flowchart of association processing in the first embodiment;

FIG. 10A is a diagram illustrating an example of an association screen;

FIG. 10B is a diagram illustrating an example of the association screen;

FIG. 11 is a diagram illustrating an example of a data display screen;

FIG. 12 is a diagram illustrating an example of the data display screen;

FIG. 13 is a diagram illustrating an example of the data display screen;

FIG. 14 is a diagram illustrating an example of the data display screen;

FIG. 15 is a block diagram of a data management device according to a second embodiment;

FIG. 16 is a block diagram of a terminal device according to the second embodiment;

FIG. 17 is a diagram illustrating an example of a screen used to print identification information;

FIG. 18 is a diagram illustrating an example of an association screen used for association;

FIG. 19 is a flowchart of association processing in the second embodiment;

FIG. 20 is a flowchart of storage processing in the second embodiment;

FIG. 21 is a diagram illustrating an example of a relationship between a plurality of culture actions and a job;

FIG. 22 is a block diagram of a data management device according to a third embodiment;

FIG. 23 is a diagram illustrating an example of a data structure of job data;

FIG. 24 is a diagram illustrating an example of an input screen configured to input job data;

FIG. 25 is a diagram illustrating an example of a display screen configured to display job data;

FIG. 26 is a diagram illustrating an example of a print screen; and

FIG. 27 is a hardware configuration diagram of the device according to the first to third embodiments.

### DETAILED DESCRIPTION

A data management system according to an embodiment includes a first storage control unit, a second storage control unit, and a third storage control unit. The first storage control unit is configured to control storing, in a storage unit, one or more culture conditions indicating conditions for culturing a cell. The second storage control unit is configured to control storing, in the storage unit, one or more pieces of measurement data that are received via a network and that are for the cell cultured based on any one of the culture conditions. The third storage control unit is configured to control storing, in the storage unit, one or more pieces of the measurement data designated among the pieces of the measurement data and one or more of the culture conditions designated among the culture conditions, in association with each other.

Hereinafter, embodiments of a data management system will be described in detail with reference to the accompanying drawings.

### First Embodiment

Cells include established cells, cells separated from biological tissues, unicellular organisms, fungi, bacteria, spheroids, organoids, cell sheets, and the like. As a method of managing measurement data such as image data of cells, for example, a method of managing measurement data by dividing the measurement data into folders for each measurement device or measurement date can be considered. In such a method, for example, as to under what culture conditions (conditions for culturing cells) each piece of measurement data is cultured, a procedure of separately generating data regarding culture conditions and managing the data in association with the corresponding folder is required. Even if the culture conditions are managed by such a procedure, only the items of the culture conditions that are of interest at that time are left in any format, and even if the items are browsed by a person other than the operator who registers the data, the readability is poor, and the items of sufficient culture conditions are not included. As a result, for example, it is not easy to analyze measurement data under different culture conditions, grasp the difference in culture conditions, or obtain appropriate culture conditions.

Therefore, a data management system according to a first embodiment realizes a data management method capable of more efficiently analyzing measurement data. The data management system according to the present embodiment manages a culture vessel as a minimum unit of a “culture action” in association with measurement data obtained in relation to the “culture action” using the culture vessel. Further, in the culture action, a unit obtained by grouping culture vessels in which at least a part of culture conditions such as a cell type and a culture date and time is common may be provided. Culture conditions are determined for the culture vessel. As such, the management method can be rephrased as a method of managing measurement data and the like with the culture condition as a minimum unit.

The culture vessel is a vessel configured to culture cells, such as a dish, a flask, and a multi-well. In the case of the multi-well, each of the plurality of holes (wells) provided in the multi-well can be interpreted as a culture vessel. Hereinafter, a unit of data for data management corresponding to the culture vessel is referred to as a container.

For example, the culture conditions include the following conditions, but are not limited thereto.
Conditions related to cells to be cultured: cell type, the number of passages, type of gene expression, vector, where to obtain, donor, cell average size, cell seeding concentration, and cell seeding density
Conditions related to vessel to be used for culture: vessel type (dish, flask, and multi-well), vessel size, coating, and vessel bottom area
Conditions related to culture medium to be used for culture: type of culture medium, amount of culture medium, culture medium additive, and serum
Conditions related to reagent to be used for culture: reagent name, reagent amount, and reagent concentration
Other conditions: registrant, operator, culture start date, culture end date, parent container, and child container

A measurement device configured to obtain measurement data may be any device. For example, the following measurement devices can be used.
Microscope: output image data
Culture monitoring device with microscopic image capturing function: output image data
Cell counter: output at least one piece of image data and cell concentration data
Measurement device such as fluorescence activated cell sorting (FACS), polymerase chain reaction (PCR), and next generation sequencing (NGS): output data indicating a measurement result

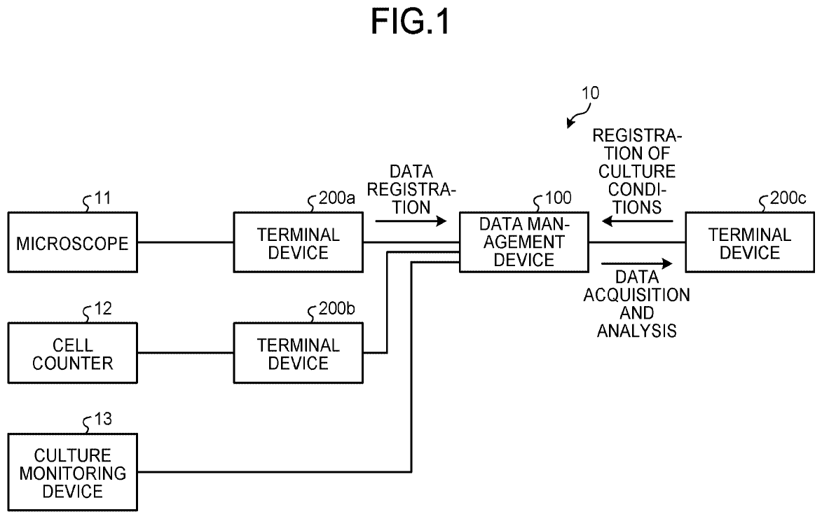

FIG. 1 is a diagram illustrating a configuration example of a data management system 10 according to the present embodiment. The data management system 10 includes measurement devices such as a microscope 11, a cell counter 12, and a culture monitoring device 13, a data management device 100, and terminal devices 200a, 200b, and 200c. Note that the number of each of the microscope 11, the cell counter 12, and the culture monitoring device 13 is not limited to one, and may be plural.

The microscope 11 is connected to the terminal device 200a, and transmits measurement data to the data management device 100 via the terminal device 200a. The cell counter 12 is connected to the terminal device 200b, and transmits measurement data to the data management device 100 via the terminal device 200b.

The culture monitoring device 13 is connected to the data management device 100 without a terminal device, and directly transmits measurement data to the data management device 100. The culture monitoring device 13 may be a device configured to measure a microscopic image of a cell or the like while culturing the cell therein. On the basis of the microscopic image measured by the culture monitoring device 13, the data management device 100 or the culture monitoring device 13 can also analyze real-time cell density, growth curve, doubling time, and the like, and use these as one piece of measurement data. Furthermore, the culture monitoring device 13 may be a device having a function of measuring at least one of temperature, CO₂ concentration, O₂ concentration, vibration, and the like, and when measurement is performed, these can also be used as one piece of the measurement data.

In this manner, the measurement device may be directly connected to the data management device 100, or may be connected to the data management device 100 via another device (the terminal devices 200a, 200b, and the like).

As a method of collecting the measurement data from the measurement device, for example, there is a method of writing the measurement data obtained by the measurement device in a storage medium such as a universal serial bus (USB) memory, reading the measurement data from the storage medium to a certain terminal device (for example, the terminal device 200c), and arranging the measurement data. However, in such a method, there is a method of executing processing such as writing measurement data to the storage medium and reading measurement data from the storage medium by the number of measurement devices, and a burden on the user is large.

On the other hand, in the data management system 10 according to the present embodiment, each measurement device transmits the measurement data to the data management device 100 via the terminal device connected thereto, or transmits the measurement data to the data management device 100 directly connected thereto. Therefore, even in a configuration including a plurality of measurement devices and a plurality of terminal devices connected to the measurement devices, measurement data can be collected more easily.

In a case where measurement data is stored in a device (such as a personal computer) other than the terminal device connected to the measurement device, the device may access the data management device 100 to transmit (upload) the measurement data to the data management device 100. For example, it may be configured such that the data management device 100 is accessed by a web browser or the like from a device in which measurement data is stored, and upload of the measurement data can be designated in a screen configured to display data for each container.

The data management device 100 and each of the terminal devices 200a, 200b, and 200c are connected to each other, for example, via a network such as the Internet. Furthermore, by configuring the terminal devices 200a, 200b, and 200c to be connectable to a wireless network line provided by a communication company, it is not necessary for a user to set network connection by himself or herself, and a side that provides and manages the data management device can remotely investigate a cause when a trouble occurs. Note that the connection form is not limited thereto, and any form may be used.

The data management device 100 is a device configured to manage measurement data in units of a container. The data management device 100 may be configured as a physically independent server device, or may be, for example, a server device logically configured on a cloud environment.

The terminal devices 200a, 200b, and 200c can be configured by, for example, a personal computer, a tablet terminal, a smartphone, or the like. The terminal devices 200a and 200b are connected to the measurement device (the microscope 11, the cell counter 12) via a network such as a local area network (LAN) or a dedicated line or the like, and transmit (register data) measurement data measured by the connected measurement device to the data management device 100. For example, the terminal devices 200a and 200b can transmit the measurement data measured by the measurement device to the data management device 100 via an USB type dongle device.

The USB type dongle device refers to a device having a USB memory function, a wireless communication function, and a transmission/reception script execution function. In a case where the measurement device has a connection function to a USB, the USB type dongle device may be connected to the measurement device, and measurement data may be transmitted to the data management device 100 via the USB type dongle device. For example, the USB type dongle device can automatically upload the measurement data to the data management device 100 upon detecting the writing of the measurement data from the connected measurement device.

The network between the measurement device and the terminal devices 200a and 200b or the data management device 100 and the network between the terminal device 200c and the data management device 100 may be either wireless or wired.

The terminal device 200c is used for registration of data such as culture conditions to the data management device 100, acquisition of measurement data from the data management device 100, and the like. One of the terminal devices 200a and 200b and the terminal device 200c may be implemented by a common device. Hereinafter, a description will be given on the assumption that the data management system 10 includes a terminal device 200 having both the functions of one of the terminal devices 200a and 200b and the terminal device 200c.

Each measurement device and the terminal device 200 are provided, for example, in a facility in which culture is performed (a university, a laboratory, and the like). For example, the data management device 100 may be provided in a place different from this facility (an installation place of a server for a cloud environment), or may be provided in the same facility as a measurement device or the like.

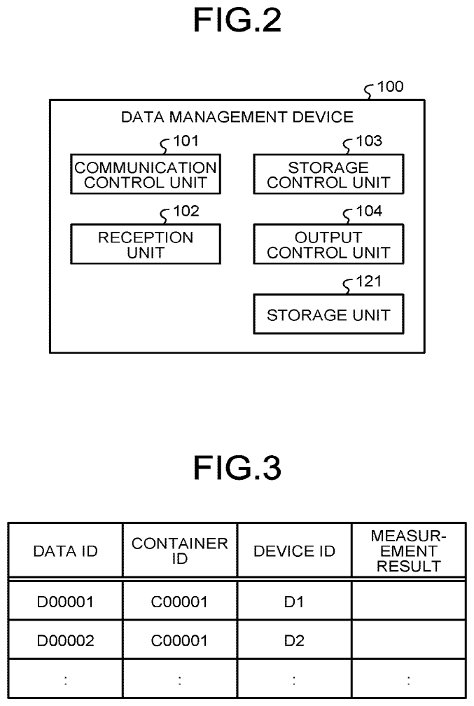

FIG. 2 is a block diagram illustrating an example of a functional configuration of the data management device 100. As illustrated in FIG. 2, the data management device 100 includes a communication control unit 101, a reception unit 102, a storage control unit 103, an output control unit 104, and a storage unit 121.

The communication control unit 101 controls communication with external devices such as the culture monitoring device 13 and the terminal device 200. For example, the communication control unit 101 receives measurement data transmitted from the culture monitoring device 13 and the terminal device 200. The communication control unit 101 receives culture conditions transmitted from the terminal device 200. The communication control unit 101 transmits measurement data requested from the terminal device 200 to the terminal device 200.

The reception unit 102 receives an input of one or more culture conditions and one or more pieces of measurement data for cells cultured based on any one of the culture conditions. For example, the reception unit 102 receives the culture conditions received from the terminal device 200. The reception unit 102 may receive culture conditions input by an input device (a keyboard, a mouse, and the like) provided in the data management device 100. The reception unit 102 receives the measurement data received from the culture monitoring device 13 and the terminal device 200.

The storage control unit 103 controls processing of storing data in the storage unit 121. For example, the storage control unit 103 controls storing one or more culture conditions in the storage unit 121 (a first storage control unit). In addition, the storage control unit 103 controls storing one or more pieces of measurement data in the storage unit 121 (a second storage control unit). In addition, the storage control unit 103 controls storing one or more pieces of designated measurement data among the received measurement data in the storage unit 121 in association with one or more designated culture conditions among the culture conditions (a third storage control unit). For example, the storage control unit 103 performs association processing based on designation input by a user using a display screen displayed on a display unit 211 (described later) of the terminal device 200 by the output control unit 104. Details of the association processing will be described later.

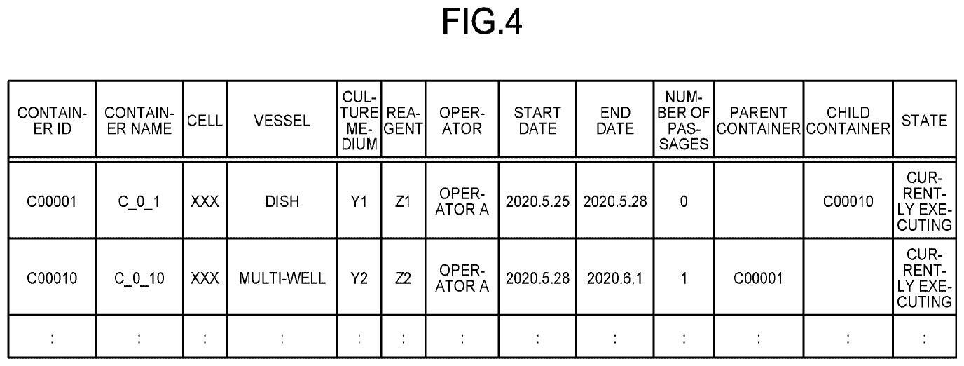

An example of a method of controlling storing data in the storage unit 121 will be described below. FIG. 3 is a diagram illustrating an example of a data structure of the measurement data to be stored in the storage unit 121. FIG. 4 is a diagram illustrating an example of a data structure of the culture conditions (containers) to be stored in the storage unit 121.

As illustrated in FIG. 3, the measurement data includes a data identifier (ID), a container ID, a device ID, and a measurement result. The data ID is information for identifying measurement data. The container ID is identification information for identifying a container. Note that no value is set to the container ID before the measurement data is associated with the culture conditions (containers). The device ID is information for identifying a measurement device. The measurement result is a result of measurement by the measurement device. A data format of the measurement result may be changed depending on the measurement device. For example, the measurement result may include image data, the counted number of cells, and the like. For the measurement result, data itself may be stored, or identification information of the measurement result or information indicating a place in which the measurement result is stored and the like may be stored.

As illustrated in FIG. 4, the container includes a container ID, a container name, one or more culture conditions (a cell, a vessel, a culture medium, and a reagent), an operator, a start date, an end date, the number of passages, a parent container, a child container, and a state. The container name represents a name of a container.

The cell, the vessel, the culture medium, and the reagent correspond to the conditions related to the cell to be cultured, the conditions related to the vessel to be used for culture, the conditions related to the culture medium to be used for culture, and the conditions related to the reagent to be used for culture. Other culture conditions may be further included.

The operator is, for example, a name of a user who generates the container. The start date and the end date are a date at which the culture is started and a date at which the culture is ended, respectively. The number of passages indicates how many times the cells corresponding to the container are passaged from other cells. The parent container represents a container ID of a container corresponding to a cell from which passage is performed when a cell of the corresponding container is a passaged cell. The child container represents a container ID of a container corresponding to another cell when another cell is passaged from a cell of the corresponding container. The state represents a state of culture of a cell (executing, finished, and the like).

The data structure of the container is not limited to the structure illustrated in FIG. 4. The container may further include information other than these pieces of information, or may not include some of these pieces of information. For example, the container may not include the container name.

Note that FIGS. 3 and 4 illustrate examples in which the measurement data includes the container ID and the device ID. However, the container illustrated in FIG. 4 may include the data ID, the measurement data illustrated in FIG. 3 may include the device ID, and the device ID of the measurement data may be stored in the data ID of the container, so that the container ID and the device ID are associated with each other. Alternatively, a relational table having the container ID, the data ID, and the device ID as registration information may be provided, and the respective ones ID may be associated with each other via the relational table.

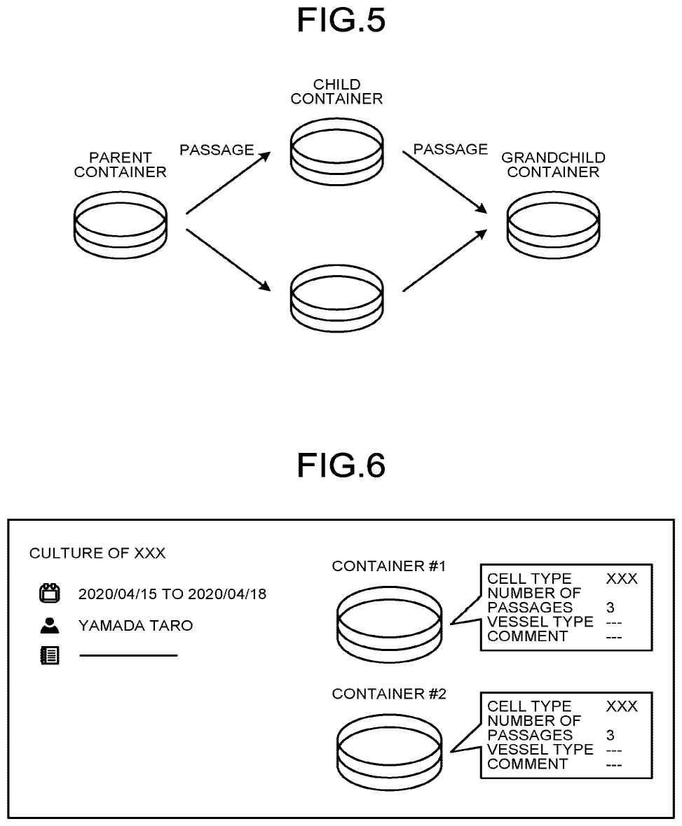

FIG. 5 is a diagram illustrating an example of passage. FIG. 5 illustrates an example in which two child containers are passaged from a parent container, and one container (a grandchild container) is further passaged from the two child containers. When there are a plurality of parent containers and when there are a plurality of child containers, a plurality of containers ID may be set in each of the parent container field and the child container field in FIG. 4. When one container is set as the parent container or the child container, the other container is set as the child container or the parent container.

That is, the storage control unit 103 controls storing a container (corresponding to a first culture condition) for a certain cell (a first cell) and a container (corresponding to a second culture condition) for a cell (a second cell) passaged from the cell in the storage unit 121 in association with each other such that a parent-child relationship is represented. By associating the containers passaged in this way, the parent-child relationship can be easily managed.

When the culture conditions are grouped and managed in the culture action, information for identifying the group may be included in the container. That is, the storage control unit 103 may classify a plurality of culture conditions at least partially matching each other into the same group and control storing the same in the storage unit 121. By grouping and managing the containers, there are effects such as improvement of the browsability for a user and easy editing of some pieces of information on the grouped containers together.

FIG. 6 is a diagram illustrating an example of grouping of culture conditions. In FIG. 6, an example of a culture action in which two containers #1 and #2 are grouped is illustrated. As illustrated in this example, when a plurality of containers is included in one culture action, a plurality of containers (culture conditions) can be grouped and managed.

Referring back to FIG. 2, the output control unit 104 controls the output of various types of data used in the data management device 100. For example, the output control unit 104 outputs a screen to be displayed on the display unit 211 (described later) of the terminal device 200. The output control unit 104 outputs, for example, screen information for selectively displaying one or more culture conditions and one or more pieces of measurement data.

Each of the above-described units (the communication control unit 101, the reception unit 102, the storage control unit 103, and the output control unit 104) is implemented by, for example, one or more processors. For example, each of the above-described units may be implemented by causing a processor such as a central processing unit (CPU) to execute a program, that is, by software. Each of the above-described units may be implemented by a processor such as a dedicated integrated circuit (IC), that is, by hardware. Each of the above-described units may be implemented by using software and hardware in combination. In the case of using a plurality of processors, each processor may implement one of the respective units, or may implement two or more of the respective units.

In the storage unit 121, various types of data is stored to be used in the data management device 100. For example, as described with reference to FIGS. 3 and 4, in the storage unit 121, a plurality of culture conditions is stored by being associated with one or more pieces of measurement data.

The storage unit 121 can be configured by any generally used storage medium such as a flash memory, a memory card, a random access memory (RAM), a hard disk drive (HDD), and an optical disc.

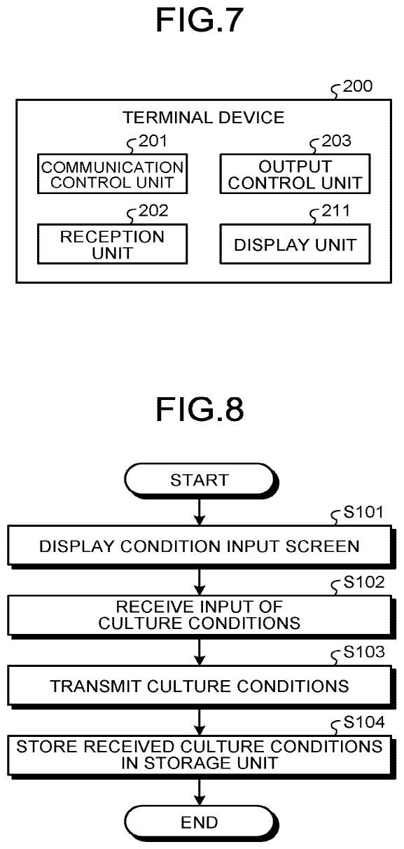

FIG. 7 is a block diagram illustrating an example of a functional configuration of the terminal device 200. As illustrated in FIG. 7, the terminal device 200 includes a communication control unit 201, a reception unit 202, an output control unit 203, and the display unit 211.

The display unit 211 is a display device such as a liquid crystal display configured to display data.

The communication control unit 201 controls communication with external devices such as the culture monitoring device 13 and the data management device 100. For example, the communication control unit 201 receives the measurement data transmitted from the culture monitoring device 13. The communication control unit 201 transmits the measurement data to the data management device 100.

The reception unit 202 receives inputs of various types of data used by the terminal device 200. For example, the reception unit 202 receives information input by a user or the like on a screen displayed on the display unit 211 by the output control unit 203.

The output control unit 203 controls outputs of various types of data used in the terminal device 200. For example, the output control unit 203 outputs a screen to be displayed on the display unit 211.

Each of the above-described units (the communication control unit 201, the reception unit 202, and the output control unit 203) is implemented by, for example, one or more processors. For example, each of the above-described units may be implemented by causing a processor such as a CPU to execute a program, that is, by software. Each of the above-described units may be implemented by a processor such as a dedicated IC, that is, by hardware. Each of the above-described units may be implemented by using software and hardware in combination. In the case of using a plurality of processors, each processor may implement one of the respective units, or may implement two or more of the respective units.

Next, condition storage processing by the data management system 10 according to the first embodiment configured as described above will be described. FIG. 8 is a flowchart illustrating an example of condition storage processing in the first embodiment. The condition storage processing is processing of controlling storing the culture conditions as illustrated in FIG. 4 in the storage unit 121 of the data management device 100.

The output control unit 104 of the data management device 100 generates information for displaying a condition input screen used to input culture conditions, and causes the display unit 211 to display the information via the output control unit 203 of the terminal device 200 (step S101). Note that the output control unit 203 of the terminal device 200 may generate a condition input screen and display the same on the display unit 211.

The condition input screen is a screen configured to input the culture conditions as described in FIG. 4. The condition input screen may have a function (an import function) of copying the same culture conditions as the already generated container to generate a new container, a function of displaying and selecting options of each condition, a function (an auto-complete function) of predicting a value to be input with reference to a past input or the like and displaying the predicted value, and the like. As a result, it is easy to record the culture conditions including detailed matters that are troublesome and difficult in the record management using the experiment notes and the data management using the folders.

In addition, the condition input screen may include, for example, a check field configured to select whether to set a container as a parent container, and in a case where the container is selected as the parent container at the time of executing an import function, a parent-child relationship may be settable between a new container and a container as a copy source. As a result, it is possible to easily generate a child container in which the number of passages is automatically increased by one from a certain container. The condition input screen may have a function (such as pop-up message display) of notifying a user that the number of passages increases. By notifying the user of the processing automatically performed by the system in this manner, there is an effect of avoiding making a mistake such as erroneously increasing the number of passages by one while reducing the labor of manual input. On the screen, the parent-child relationship may be displayed, for example, in a tree shape.

The reception unit 202 receives the culture conditions input on the condition input screen (step S102). The communication control unit 201 transmits the received culture conditions to the data management device 100 (step S103). The transmitted culture conditions are received by the communication control unit 101 and received by the reception unit 102.

The storage control unit 103 controls storing the received culture conditions in the storage unit 121 (step S104), and ends the condition storage processing.

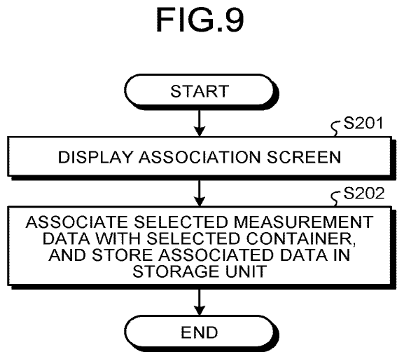

Next, association processing will be described. FIG. 9 is a flowchart illustrating an example of association processing in the first embodiment. The association processing is, for example, processing of associating one or more culture conditions to be stored in the condition storage processing with measurement data obtained from each measurement device.

A plurality of pieces of measurement data may be associated with one container (a culture condition). For example, a plurality of pieces of measurement data may be acquired for one container as follows.
Image data captured at the start
Image data captured from start to end
Image data captured at the end
Measurement data of the number of cells counted at the start
Measurement data of the number of cells counted at the end
Monitoring data captured by culture monitoring device

In addition, one piece of measurement data may be associated with a plurality of containers (culture conditions). For example, if a cell suspension adjusted at a certain concentration (density of cells) is seeded into a plurality of containers, one piece of data of the cell counter 12 is associated with a plurality of containers.

As described above, in culturing cells, measurement data and culture conditions (containers) may be associated in a many-to-many manner. The present embodiment provides an interface capable of controlling storing measurement data and culture conditions in association with each other so as to represent such a many-to-many relationship, and capable of more efficiently associating many-to-many information.

First, the output control unit 104 of the data management device 100 generates information for displaying an association screen used to associate the measurement data with the culture conditions, and causes the display unit 211 to display the information via the output control unit 203 of the terminal device 200 (step S201). Note that the output control unit 203 of the terminal device 200 may generate an association screen and display the association screen on the display unit 211.

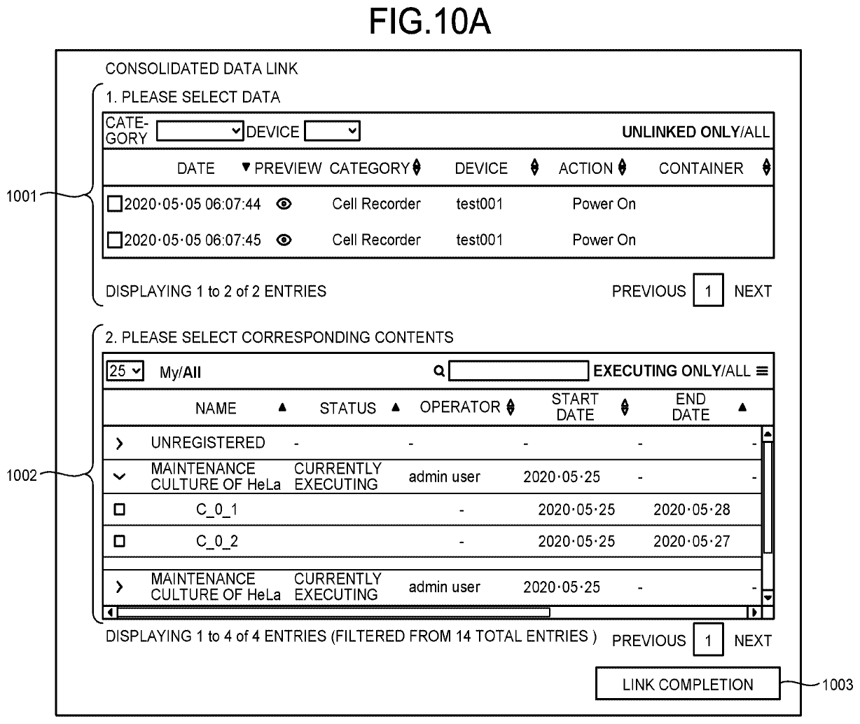

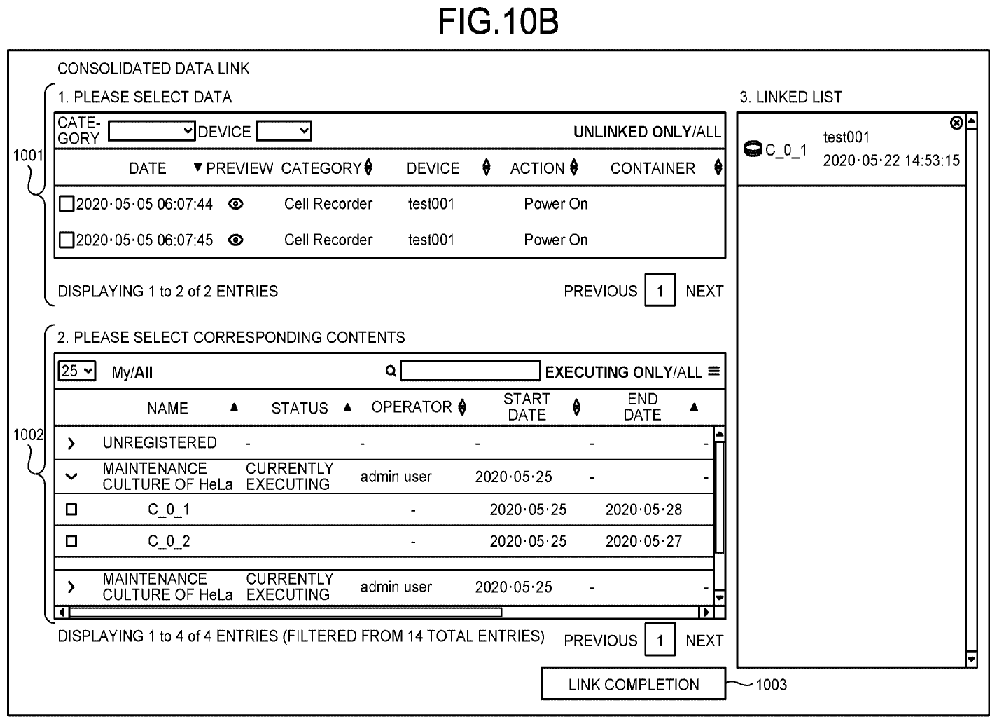

FIGS. 10A and 10B are diagrams illustrating examples of an association screen. As illustrated in FIGS. 10A and 10B, the association screen includes a measurement data selection field 1001 and a container selection field 1002.

In the selection field 1001, for example, measurement data to be stored in the storage unit 121 is displayed in a list format so as to be selectable. A function of searching for measurement data to be displayed in the selection field 1001 of the association screen among the measurement data to be stored in the storage unit 121 in the association screen or in the screen before transitioning to the association screen may be provided.

For example, the selection field 1001 includes a pull-down for selecting a category of a measurement device. The measurement device belonging to the category selected by this pull-down can be selected by a pull-down for further selecting a measurement device. When the measurement device is selected, measurement data measured by the selected measurement device is displayed in a list in the selection field 1001.

In the selection field 1002, for example, containers to be stored in the storage unit 121 are displayed in a list format so as to be selectable. Similarly to the selection field 1001, a function of searching for a container to be displayed in the selection field 1002 of the association screen among the containers to be stored in the storage unit 121 in the association screen or in the screen before transitioning to the association screen may be provided.

When a link completion button 1003 is pressed, the storage control unit 103 associates one or more pieces of measurement data selected in the selection field 1001 with one or more containers selected in the selection field 1002, and controls storing the associated data in the storage unit 121 (step S202). For example, in a case where the data structures as illustrated in FIGS. 3 and 4 are used, the container ID of the container selected in the selection field 1002 is set in the container ID field of FIG. 3.

FIG. 10B is different from FIG. 10A in that a further linked list is displayed. In this list, data associated by the selection field 1001 and the selection field 1002 is displayed as a list. By displaying such a list, a user can easily execute processing such as confirmation of the association status and re-association in accordance with the confirmation result.

An association method is not limited thereto, and any method may be used as long as one or more pieces of measurement data and one or more containers (culture conditions) are associated with each other. For example, the storage control unit 103 may receive a container (a culture condition) input by the user with respect to the measurement data selected by the user, and control storing the received container and the selected measurement data in association with each other.

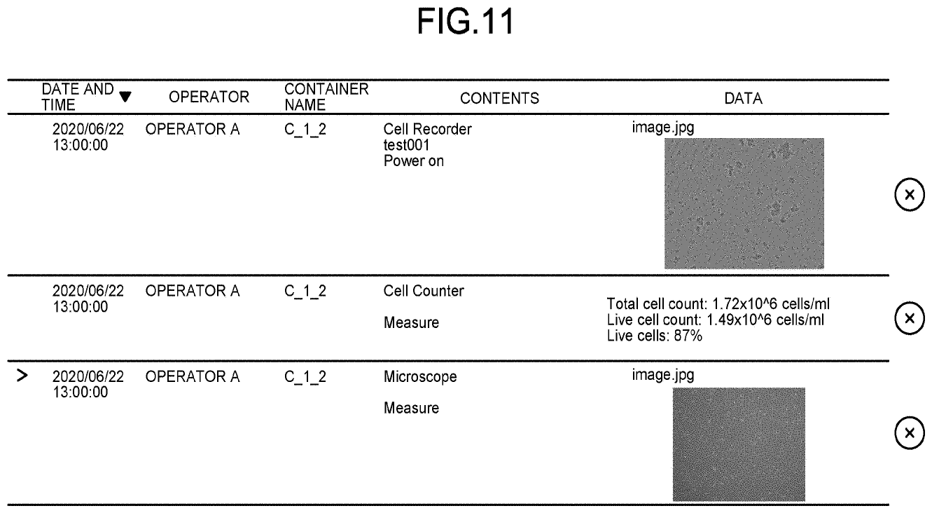

The data to be stored in this manner can be displayed on a screen by various methods and used for analysis and the like. FIGS. 11 to 14 are diagrams illustrating examples of screens configured to display data. The output of each screen is controlled by, for example, the output control unit 104.

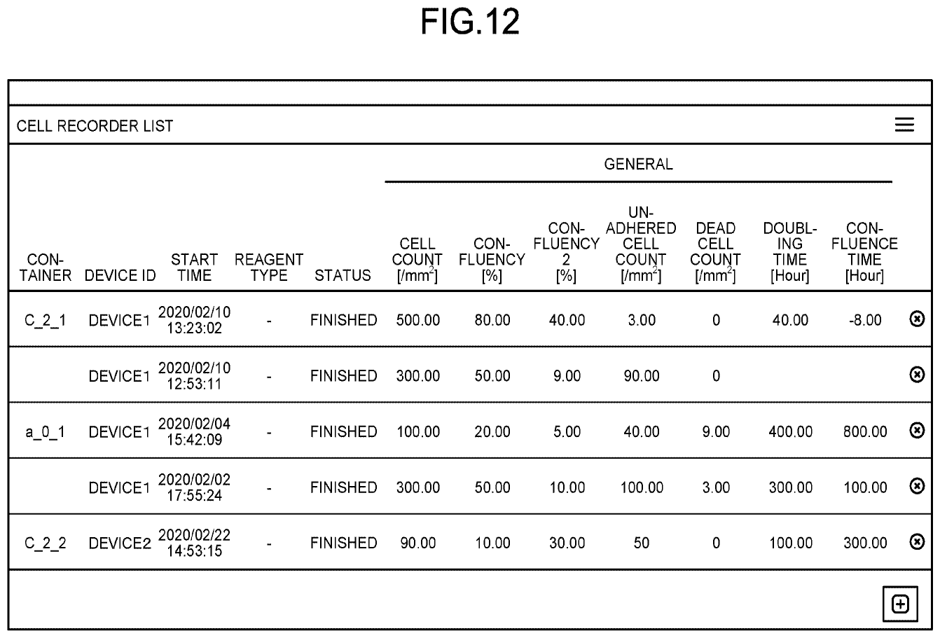

FIG. 11 is an example of a screen configured to display a plurality of pieces of measurement data associated with a certain container in a list format. FIG. 12 is an example of a screen configured to display one or more pieces of measurement data measured by a certain measurement device (a culture monitoring device) for a plurality of containers in a list format.

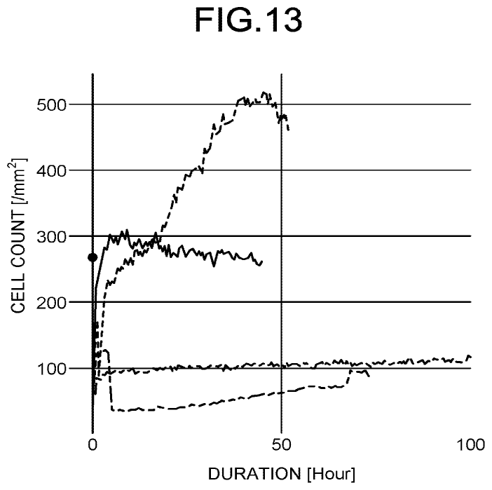

FIG. 13 is an example of a screen configured to display the correspondence between the duration and the cell count for a plurality of containers using a graph. Four lines in FIG. 13 respectively correspond to different containers (culture conditions).

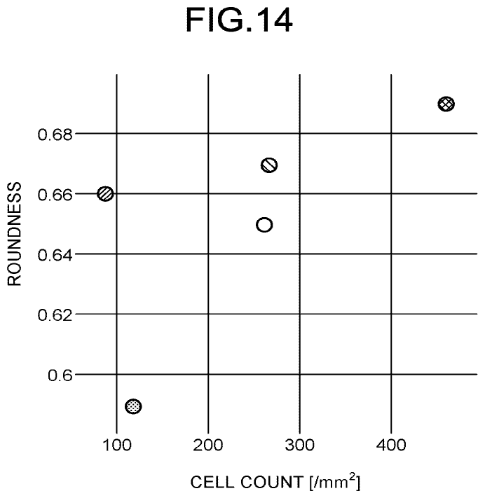

FIG. 14 is an example of a scatter diagram illustrating a relationship between the cell count and the roundness for a plurality of containers.

As described above, for example, the output control unit 104 can output one or more pieces of measurement data associated with a plurality of culture conditions (containers) to be stored in the storage unit 121 in a comparable manner.

As described above, in the data management system according to the first embodiment, management is performed in association with measurement data in units of a culture vessel corresponding to one or more culture conditions. Accordingly, it is possible to efficiently analyze the measurement data. For example, it is possible to more efficiently analyze the measurement data under different culture conditions, grasp the difference in culture conditions, or obtain appropriate culture conditions. In addition, by managing cell passage, differences obtained from data between passages can be more easily analyzed.

(Modification)

Association between the measurement data and the container is not limited to a method using a screen. For example, a measurement device may assign a container ID to a file name of measurement data, and the storage control unit 103 may identify a container by the container ID assigned to the transmitted file name of the measurement data, and associate the identified container with the measurement data. The container ID assigned by the measurement device is designated by, for example, a user. Note that the present modification can also be applied to the following embodiments.

Alternatively, a file name of measurement data may be freely generated by a user, a container that may be associated with the measurement data may be suggested to the user as a candidate from the containers to be stored in the storage unit 121 using information such as a cell type, a vessel, a culture medium, a reagent, and the number of passages included in the file name generated by the user as a key, and association may be performed by allowing the user to make a selection. For example, the storage control unit 103 generates a database based on information such as the cell type, the vessel, the culture medium, the reagent, and the number of passages included in the container to be stored in the storage unit 121, and controls storing the database in the storage unit 121. The storage control unit 103 uses the file name of the measurement data received by the reception unit 102 to search the database using a method such as “fuzzy search” and “partial match search”. The output control unit 104 displays a container obtained as a search result on the display unit 211 as a candidate. The storage control unit 103 associates the measurement data with the container according to the user's selection received by the reception unit 202.

### Second Embodiment

In the first embodiment, an example is described in which measurement data and culture conditions are to be stored in the storage unit 121 of the data management device 100 and then the measurement data and the culture conditions are associated with each other. The association may be performed on a terminal device side. In a second embodiment, an example in which a function of performing association on a terminal device side is further provided will be described. Note that it is also possible to configure such that the association is performed only on the terminal device side and the association is not performed on the data management device side.

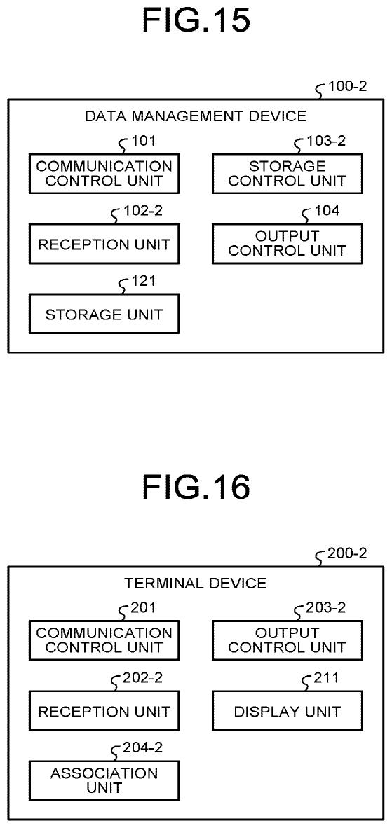

FIG. 15 is a block diagram illustrating an example of a configuration of a data management device 100-2 according to the second embodiment. As illustrated in FIG. 15, the data management device 100-2 includes a communication control unit 101, a reception unit 102-2, a storage control unit 103-2, an output control unit 104, and a storage unit 121.

In the second embodiment, functions of the reception unit 102-2 and the storage control unit 103-2 are different from those in the first embodiment. Since other configurations and functions are similar to those in FIG. 2 that is a block diagram of the data management device 100 according to the first embodiment, the same will be denoted by the same reference numerals, and a description thereof will be omitted here.

The reception unit 102-2 is different from the reception unit 102 of the first embodiment in that the reception unit 102-2 receives information associating culture conditions with measurement data from a terminal device 200-2 (described later). The storage control unit 103-2 is different from the storage control unit 103 in the first embodiment in that the storage control unit 103-2 further has a function of controlling storing culture conditions and measurement data in association with each other in the storage unit 121 according to the received information.

FIG. 16 is a block diagram illustrating an example of a configuration of the terminal device 200-2 according to the second embodiment. As illustrated in FIG. 16, the terminal device 200-2 includes a communication control unit 201, a reception unit 202-2, an output control unit 203-2, an association unit 204-2, and a display unit 211.

The second embodiment is different from the first embodiment in that the association unit 204-2 is added and functions of the reception unit 202-2 and the output control unit 203-2 are provided. Since other configurations and functions are similar to those in FIG. 7 that is a block diagram of the terminal device 200 according to the first embodiment, the same will be denoted by the same reference numerals, and a description thereof will be omitted here.

An association unit 204-2 associates one or more designated culture conditions with one or more pieces of designated measurement data. The association method may be any method. Hereinafter, a method of pre-assigning identification information for identifying a culture condition (container) to a culture vessel will be described.

The output control unit 203-2 is different from the output control unit 203 in the first embodiment in that the output control unit 203-2 further has a function of outputting identification information of culture conditions (containers) to a recording medium. For example, the output control unit 203-2 outputs identification information represented by code information such as a quick response (QR) code (registered trademark) or a barcode to a recording medium such as a label paper or an IC chip.

The reception unit 202-2 is different from the reception unit 202 of the first embodiment in that the reception unit 202-2 further has a function of receiving an input of measurement data from a measurement device and a function of receiving identification information read from a recording medium by a barcode reader (not illustrated) or the like provided in the terminal device 200-2.

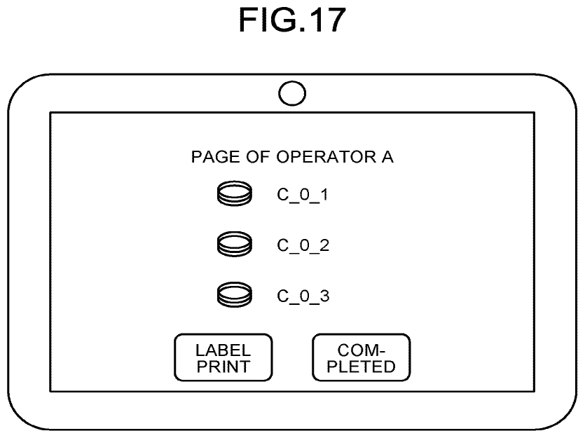

FIG. 17 is a diagram illustrating an example of a screen used to print identification information. Note that FIG. 17 illustrates an example of a screen in a case where the terminal device 200-2 is configured as a tablet terminal. As illustrated in FIG. 17, the output control unit 203-2 displays a screen including a list of containers in which identification information is to be printed on the display unit 211. The information on a target container may be input in the terminal device 200-2, or may be configured to receive the information input by the data management device 100-2.

When a label print button is pressed on the screen of FIG. 17, the identification information of each container is coded and printed on the label paper. Note that the container to be printed may be configured to be selectively displayed.

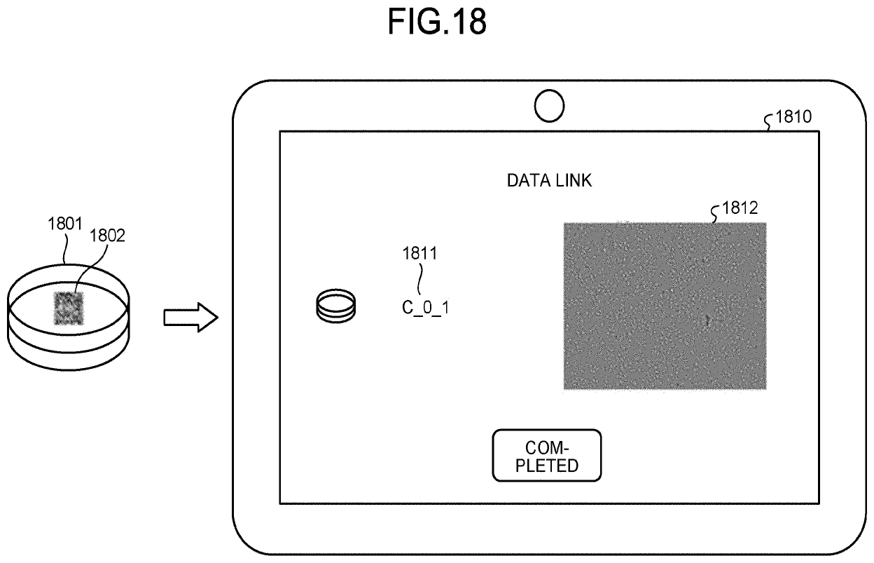

The recording medium is attached to a corresponding culture vessel. The association unit 204-2 associates culture conditions with measurement data as follows using the recording medium attached to the culture vessel. FIG. 18 is a diagram illustrating an example of an association screen used for association.

As illustrated in FIG. 18, for example, a label paper 1802 having identification information printed thereon is attached to a culture vessel 1801 by a user. The user starts culturing cells using the culture vessel 1801 to which the label paper 1802 is attached.

When measurement data for the culture vessel 1801 is measured, for example, the reception unit 202-2 inputs the measurement data into the terminal device 200-2. The user designates the measurement data and designates reading of the identification information given to the culture vessel corresponding to the designated measurement data. The association unit 204-2 associates one or more culture conditions designated in this manner with one or more pieces of designated measurement data.

FIG. 18 illustrates an example of a confirmation screen 1810 (e.g., a culture vessel 1811, a label paper 1812) configured to confirm the associated culture condition (container) and measurement data. When a completion button is pressed on the confirmation screen 1810, the associated data is transmitted to, for example, the data management device 100-2. The associated data may be stored in a storage unit or the like in the terminal device 200-2, and then the stored data may be transmitted to the data management device 100-2 according to designation of transmission or the like.

As described above, the association unit 204-2 associates the culture condition (container) identified by the identification information read from the recording medium in which the culture condition is recorded with the measurement data for the cell cultured in the culture vessel to which the recording medium is attached. The storage control unit 103-2 of the data management device 100-2 controls storing the culture conditions and the measurement data associated by the association unit 204-2 in the storage unit 121.

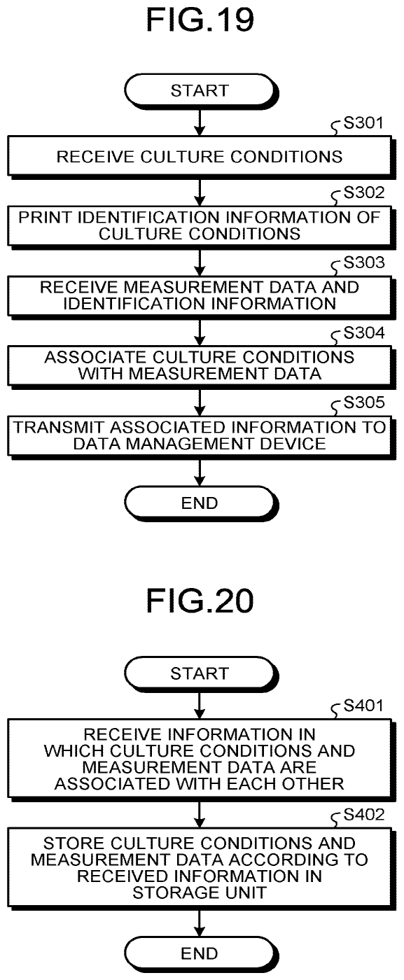

Next, association processing by the terminal device 200-2 according to the second embodiment configured as described above will be described with reference to FIG. 19. FIG. 19 is a flowchart illustrating an example of the association processing in the second embodiment.

The reception unit 202-2 receives an input of culture conditions (step S301). The output control unit 203-2 prints identification information of the culture conditions on a recording medium (step S302). The recording medium on which the identification information is printed is attached to, for example, a culture vessel by a user. Thereafter, cells are cultured using the culture vessel.

The reception unit 202-2 receives an input of measurement data with respect to the culture vessel, and receives an input of the identification information read from the recording medium attached to the culture vessel (step S303).

The association unit 204-2 associates the received measurement data with the culture conditions identified by the identification information (step S304). The communication control unit 201 transmits the associated information to the data management device 100-2 (step S305).

In the data management device 100-2, processing of controlling storing information in the storage unit 121 is executed based on the information transmitted as described above. FIG. 20 is a flowchart illustrating an example of storage processing in the second embodiment.

The communication control unit 101 receives the information in which the measurement data and the culture conditions are associated with each other transmitted from the terminal device 200-2 (step S401). The storage control unit 103-2 controls storing the culture conditions and the measurement data in the storage unit 121 in association with each other according to the received information (step S402).

As described above, in the second embodiment, the association processing can be executed on the terminal device side.

### Third Embodiment

In the culturing of cells, various kinds of work are executed by a user (person in charge, i.e., operator) in relation to a culture action. Hereinafter, such work is referred to as a job. For example, there are the following types of jobs.
Seeding of cells
Measurement by measurement device (including confirmation by the microscope 11 and the like)
Culture medium replacement
Passage

Note that these types of work may be further divided into finer elements and treated as a job. Examples of such work include washing of cells in a buffer solution, pipetting, centrifugation, and addition of a reagent such as trypsin.

In a case where there are an instruction giver for the job and a worker of the job for one culture, for the instruction giver, easy grasping is enabling considering and giving instructions for the job for each culture action (a container). On the other hand, for the worker, easy grasping is visibility of the job to be performed on that day across a plurality of culture actions (containers). However, for example, when a large number of culture actions (a unit corresponding to a container) are executed in parallel, grasping jobs to be executed on a certain day without omission is not easy.

As a method of managing the job, for example, there is a method of dividing pages of experiment notes and recording different culture actions of the same timeframe. However, in such a method, it is necessary to search pages for work to be executed on a certain day. As the number of culture actions increases, job management becomes more difficult.

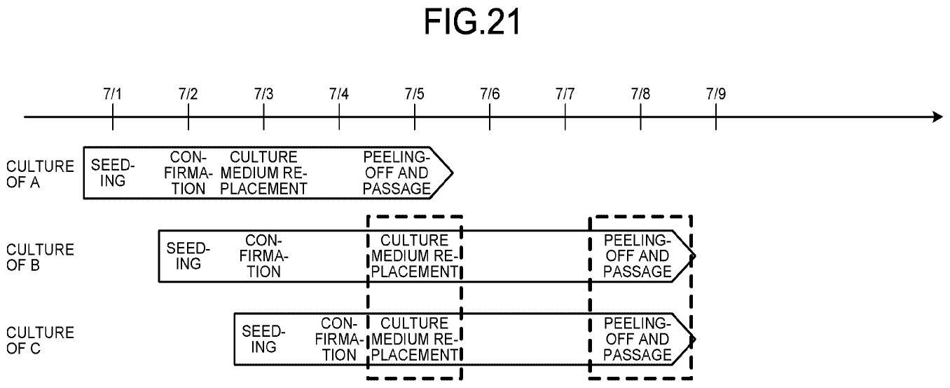

In addition, for each of the plurality of culture actions, the same job may be set as a job to be executed at the same timeframe. FIG. 21 is a diagram illustrating an example of a relationship between a plurality of culture actions and a job. In the example of FIG. 21, for a second culture action (culture of B) and a third culture action (culture of C), culture medium replacement, and peeling-off and passage are scheduled to be executed at the same timeframe, respectively.

However, when the job to be commonly executed cannot be easily grasped in this way, a wasteful work such as preparation work for each culture action may occur. For example, when the work is performed in the following order, preparation work for the culture medium replacement occurs twice.

(1) Culture medium replacement for culture of B

(2) Peeling-off and passage for culture of A

(3) Culture medium replacement for culture of C

On the other hand, for example, if the order of execution of (1) and (2) is exchanged, and culture medium replacement for culture of B and culture of C is executed as a series of work, it is possible to reduce waste of preparation work and to suppress consumption of tools (such as disposable pipettes) used for the work. In order to determine the appropriate execution order in this manner, it is desirable to provide an interface capable of efficiently managing a job for each culture action and efficiently confirming a job for one or more culture actions.

Therefore, in the third embodiment, an example of a data management system further having a job management function of managing a job will be described. In the third embodiment, a function of a data management device is different from that in the above embodiment. Other devices are similar to those in the above embodiment, and thus detailed description thereof is omitted. Note that an example in which the data management device according to the first embodiment is changed to the data management device according to the present embodiment will be described below. The data management device according to the second embodiment may be changed to the data management device according to the present embodiment.

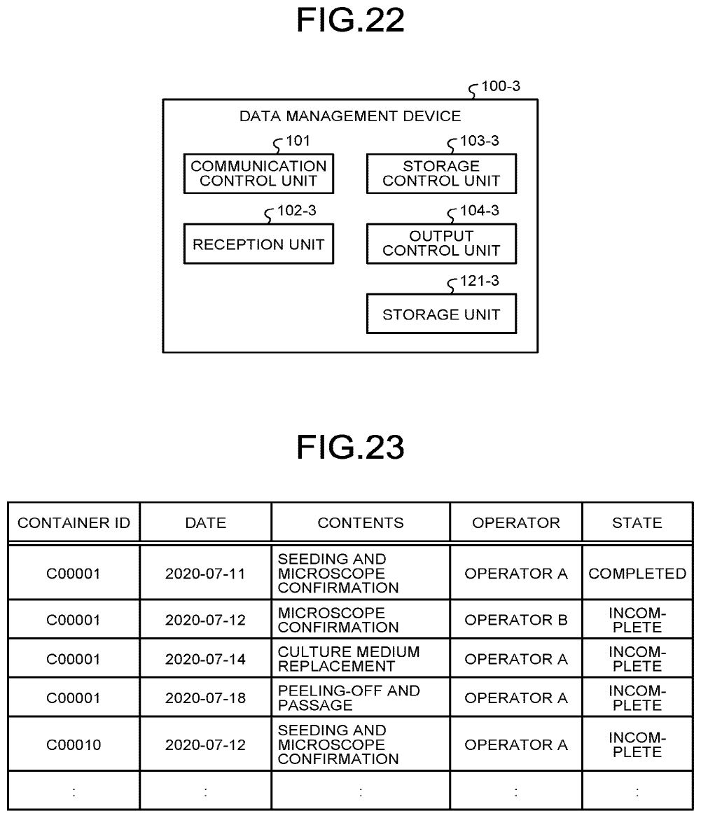

FIG. 22 is a block diagram illustrating an example of a configuration of a data management device 100-3 according to the third embodiment. As illustrated in FIG. 22, the data management device 100-3 includes a communication control unit 101, a reception unit 102-3, a storage control unit 103-3, an output control unit 104-3, and a storage unit 121-3.

In the third embodiment, functions of the reception unit 102-3, the storage control unit 103-3, the output control unit 104-3, and the storage unit 121-3 are different from those in the first embodiment. Since other configurations and functions are similar to those in FIG. 2 that is a block diagram of the data management device 100 according to the first embodiment, the same will be denoted by the same reference numerals, and a description thereof will be omitted here.

The storage unit 121-3 is different from the storage unit 121 in the first embodiment in that the storage unit 121-3 is configured to further store job data for managing jobs. FIG. 23 is a diagram illustrating an example of a data structure of job data to be stored in the storage unit 121-3.

As illustrated in FIG. 23, the job data includes a container ID, a date, a content, an operator, and a state. The container ID is a container ID of a container corresponding to a culture action for executing a job. The date indicates a work date on which the job is executed. The content indicates a content of the job. The state indicates a state of execution of the job. For example, “incomplete” is set when the execution of the job is not completed, and “completed” is set when the execution of the job is completed.

The reception unit 102-3 is different from the reception unit 102 in the first embodiment in that the reception unit 102-3 further receives inputs of various types of data related to job management. For example, the reception unit 102-3 receives an input of job data stored in the storage unit 121-3. The reception unit 102-3 also receives various types of information input by a user on various screens (described later) related to the job.

The storage control unit 103-3 is different from the storage control unit 103 in the first embodiment in that the storage control unit 103-3 further has a function of controlling storing data related to job management in the storage unit 121-3. For example, the storage control unit 103-3 controls storing the job data received by the reception unit 102-3 in the storage unit 121-3.

The output control unit 104-3 is different from the output control unit 104 in the first embodiment in that the output control unit 104-3 further has a function of controlling output of data related to job management. For example, the output control unit 104-3 outputs a screen configured to display various screens (described later) related to the job on the display unit 211 of the terminal device 200.

Next, an example of a screen used for job management will be described. Note that the following screen is an example, and any other screen may be used as long as similar information can be displayed and/or input.

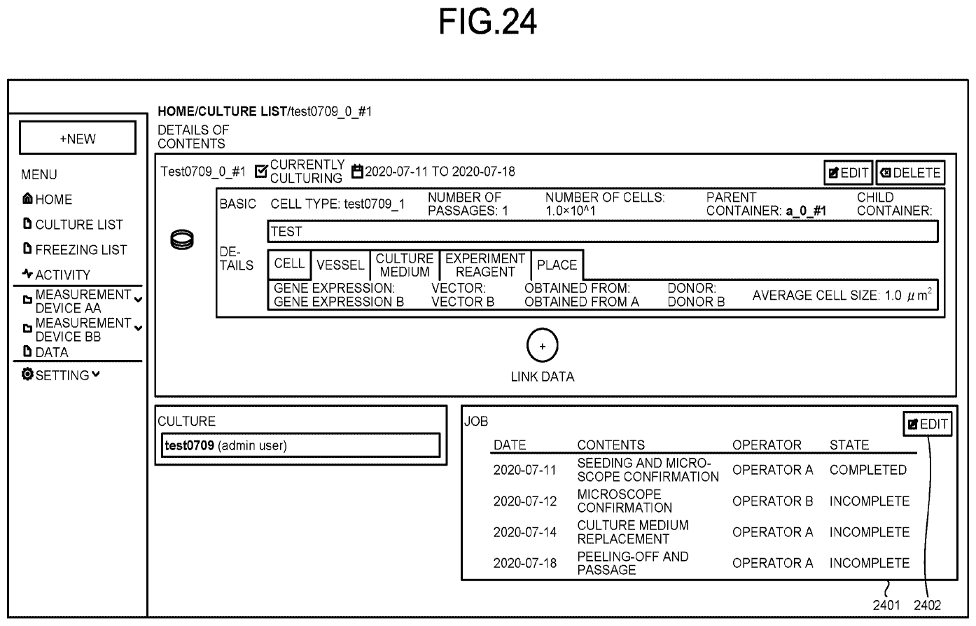

FIG. 24 is a diagram illustrating an example of an input screen configured to input job data. The input screen of FIG. 24 is an example of an input screen configured to display details of a certain container and to be able to input job data to the container.

As illustrated in FIG. 24, the input screen includes an input field 2401 configured to input job data. In the input field 2401, for example, when an edit button 2402 is pressed, a date of execution of a job, a content of a job, an operator, and a state can be input.

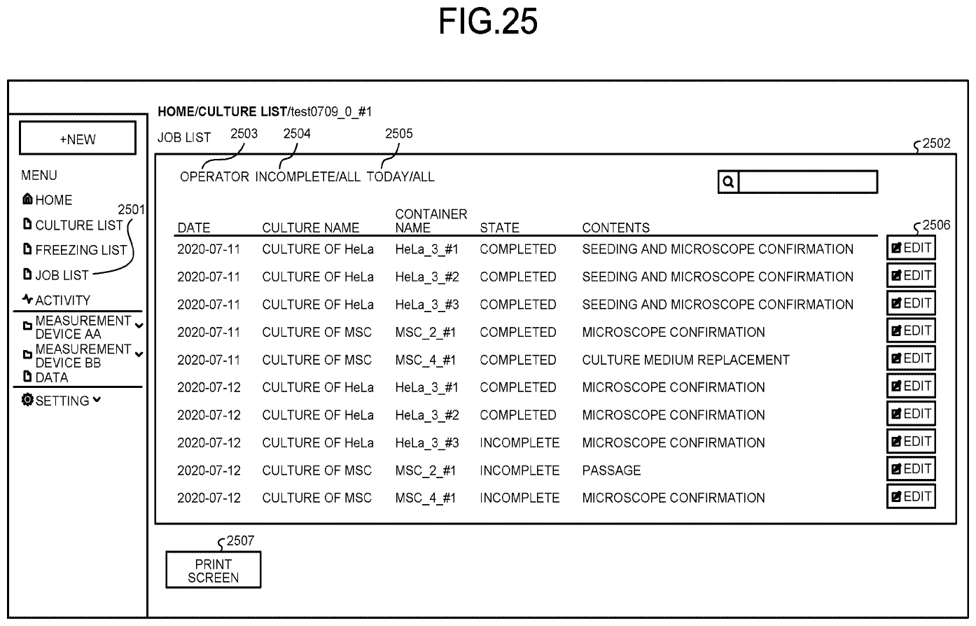

FIG. 25 is a diagram illustrating an example of a display screen configured to display input job data. As illustrated in FIG. 25, the display screen includes a display field 2502 configured to display a list of job data. The display field 2502 is displayed, for example, by selecting a job list link 2501 from a menu.

In the display field 2502, conditions of job data to be displayed can be designated. For example, when an operator designation field 2503 is selected, a pull-down menu configured to designate an operator, a designation screen, and the like are displayed. The job data of the operator designated in the pull-down menu or the designation screen is listed in the display field 2502. Note that a default value in the operator designation field 2503 may be, for example, a user using the display screen (a logged-in user or the like).

Similarly, by using a state designation field 2504 and a date designation field 2505, it is possible to designate conditions of the state and the date of the job data to be displayed in the display field 2502.

When an edit button 2506 is pressed, an edit screen (not illustrated) configured to edit (update) the job data is displayed. A user can update each item of the job data on the edit screen. For example, the reception unit 102-3 receives changed job data. The storage control unit 103-3 updates the job data in the storage unit 121-3 with the changed job data.

The display format of the job data is not limited to the list format as illustrated in FIG. 25. For example, a display screen on which an execution schedule of a job can be more easily grasped, such as a gantry chart, may be displayed. For example, as illustrated in FIG. 21, the display screen may be a screen on which jobs of a plurality of containers (culture actions) can be compared. As a result, it is possible to easily grasp a job executed in common by a plurality of culture action.

As described with reference to FIG. 24 (the edit button 2402) and FIG. 25 (the edit button 2506), the job data can be updated on the screen. A method of updating the job data is not limited thereto. For example, updated data added to a medium (a paper medium or the like) on which job data is printed may be read by an optical character reader (OCR) function or the like, and the read data may be stored in the storage unit 121-3. As a result, it is possible to solve trouble of inputting the recorded content handwritten by the user into the system and the problem of erroneous input.

For example, when a print screen button 2507 in FIG. 25 is pressed, the output control unit 104-3 generates screen information of a print screen configured to print job data, and displays the screen information on, for example, the display unit 211 of the terminal device 200.

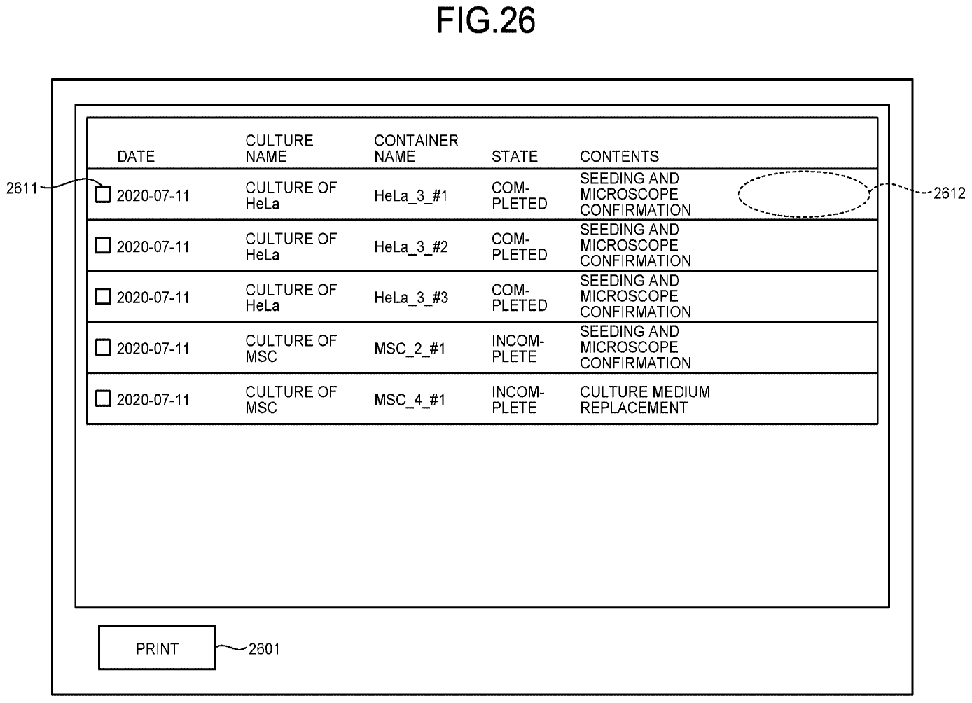

FIG. 26 is a diagram illustrating an example of a print screen. When a print button 2601 is pressed on the print screen, for example, the output control unit 104-3 causes a printer connected to the terminal device 200 to output a paper medium on which the print screen is printed.

For example, when the execution of a certain job is completed, a user writes a check mark in a check field 2611 of the corresponding job on the printed paper medium. When the content of the job is changed, the user may write the changed work in a margin 2612.

The user reads the written paper medium with an OCR device or the like, and causes the data management device 100-3 to take in the written data. The OCR device recognizes, for example, a container name, whether or not the check field 2611 is checked, a content, and an added content (a content after change). The OCR device is connected to the data management device 100-3 via a network, and transmits the read data to the data management device 100-3.

The reception unit 102-3 of the data management device 100-3 receives the transmitted data. The storage control unit 103-3 updates the job data stored in the storage unit 121-3 with the received data. For example, the storage control unit 103-3 identifies a container by the container name included in the received data, and further identifies a job by the content. The storage control unit 103-3 updates the state and content of the storage unit 121-3 according to the presence or absence of the check and the content after change included in the received data for the identified job. For example, when the presence or absence of check indicates the presence of check, the storage control unit 103-3 updates the state of the corresponding job to “completed”. In addition, in a case where the content after change is recognized, the storage control unit 103-3 updates the content of the corresponding job with the content after change.

In the above embodiment, it is assumed that the container can be identified by the container name. In a case where the container cannot be identified by the container name, for example, a container ID may be printed on a paper medium as information for identifying the container and used instead of the container name.

Characters having a size larger than the display screen (for example, FIG. 25) may be printed so that recognition by the OCR device can be executed with higher accuracy, or each line may be printed in an easily distinguishable manner. A mode in which each line is easily distinguished means, for example, that printing is performed so that each line has a different background color.

Note that the terminal device 200 may have a function of outputting a screen. For example, the output control unit 203 of the terminal device 200 may further have a function similar to that of the output control unit 104-3. For example, the output control unit 203 displays the input screen (FIG. 24), the display screen (FIG. 25), the print screen (FIG. 26), and the like on the display unit 211 of the terminal device 200. For example, the information input on the input screen is transmitted to the data management device 100-3 via the communication control unit 201.

The method of updating the job data is not limited thereto. For example, the job state may be updated using a medium (a paper medium or the like) on which information such as a barcode for updating the state is printed. For example, the output control unit 104-3 assigns a barcode for each job and prints the barcode on a paper medium. The output control unit 104-3 may print the barcode of one job on one paper medium or may print the barcodes of a plurality of jobs on one paper medium.

For example, when a user completes execution of a certain job, a barcode reading device (a barcode reader) reads a barcode of the completed job among the barcodes printed on the paper medium. The reading device is connected to the data management device 100-3 via a network, and transmits information on the read barcode to the data management device 100-3.

The reception unit 102-3 of the data management device 100-3 receives the transmitted barcode information. The storage control unit 103-3 identifies the job from the received information of the barcode, and updates the state of the identified job to “completed”.

In a case where the executed job is a job of measuring measurement data, the storage control unit 103-3 may store the measured measurement data in association with the job together with reading of the barcode.

For example, when measurement is performed by a user, a measurement device transmits the measurement data to the data management device 100 via a connected terminal device or directly. The reading device reads a barcode of a job before or after measurement according to the operation of a user, and transmits information on the read barcode to the data management device 100-3. For example, the storage control unit 103-3 of the data management device 100-3 determines that measurement data having a difference between the received times within a certain value is related to a job corresponding to a barcode, associates the measurement data as measurement data for a container corresponding to the related job, and controls storing the associated measurement data in the storage unit 121-3. In addition, as another example, a measurement device may assign job identification information or a container ID to a file name of measurement data, and the storage control unit 103-3 may identify a job by the job identification information or the container ID assigned to the file name of the transmitted measurement data, and may associate the identified job with the measurement data. The job identification information or the container ID assigned by the measurement device is designated by, for example, a user.

Alternatively, a file name of measurement data may be freely generated by the user, and a job that may be associated with the measurement data may be suggested to the user as a candidate from the jobs stored in the storage unit 121-3 using information (seeding of cells, measurement by measurement device, culture medium replacement, passage, and the like) indicating a job included in the file name generated by the user as a key, and association may be performed by allowing the user to make a selection. For example, the storage control unit 103-3 generates a database based on the information indicating the job to be stored in the storage unit 121-3, and controls storing the database in the storage unit 121-3. The storage control unit 103-3 uses the file name of the measurement data received by the reception unit 102-3 to search the database using a method such as “fuzzy search” and “partial match search”. The output control unit 104-3 displays the job obtained as a search result on the display unit 211 as a candidate. The storage control unit 103-3 associates the measurement data with the job according to the user's selection received by the reception unit 202.

The output control unit 104-3 may further have a function of performing notification related to a job. For example, the output control unit 104-3 gives a notification to an operator of the job or the like when at least one of the date and the state satisfies a predetermined condition. The notification method may be any method, and for example, a method of displaying notification on a screen and a method of giving a notification via a network (such as notification by e-mail) can be applied. The condition is, for example, a condition that the date is the current day and the state is incomplete.

As described above, in the data management system according to the third embodiment, it is possible to efficiently manage a job executed for a culture action.

As described above, according to the first to third embodiments, since the culture conditions and the measurement data are managed in association with each other, the measurement data can be efficiently analyzed.

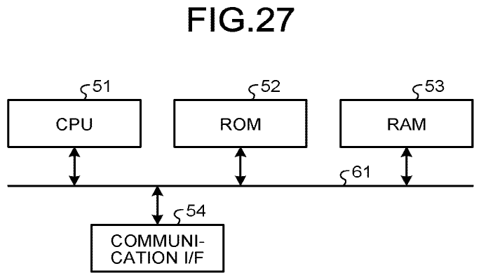

Next, a hardware configuration of the devices (the data management device and the terminal device) according to the first to third embodiments will be described with reference to FIG. 27. FIG. 27 is a hardware configuration diagram of the devices according to the first to third embodiments.

The devices according to the first to third embodiments include a control device such as a CPU 51, a storage device such as a read only memory (ROM) 52 and a random access memory (RAM) 53, a communication I/F 54 connected to a network and configured to perform communication, and a bus 61 configured to connect the respective units.

A program executed by the devices according to the first to third embodiments is provided by being incorporated in the ROM 52 or the like in advance.

The program executed by the device according to the first to third embodiments may be provided as a computer program product by being recorded in a computer-readable recording medium such as a compact disk read only memory (CD-ROM), a flexible disk (FD), a compact disk recordable (CD-R), or a digital versatile disk (DVD) as a file in an installable format or an executable format.

Furthermore, the program executed by the devices according to the first to third embodiments may be stored on a computer connected to a network such as the Internet and provided by being downloaded via the network. In addition, the program executed by the devices according to the first to third embodiments may be provided or distributed via a network such as the Internet.

The program executed by the devices according to the first to third embodiments can cause a computer to function as each unit of the above-described devices. In this computer, the CPU 51 can read a program from a computer-readable storage medium onto a main storage device and execute the program.

While certain embodiments have been described, these embodiments have been presented by way of example only, and are not intended to limit the scope of the inventions. Indeed, the novel methods and systems described herein may be embodied in a variety of other forms; furthermore, various omissions, substitutions and changes in the form of the methods and systems described herein may be made without departing from the spirit of the inventions. The accompanying claims and their equivalents are intended to cover such forms or modifications as would fall within the scope and spirit of the inventions.

What is claimed is:

1. A data management system comprising:
one or more processors coupled to a memory and configured to:
control storing, in the memory, one or more culture conditions indicating conditions for culturing a cell;
control storing, in the memory, one or more pieces of measurement data that are received via a network and that are for the cell cultured based on any one of the culture conditions; and
control storing, in the memory, one or more pieces of the measurement data designated among the pieces of the measurement data and one or more of the culture conditions designated among the culture conditions, in association with each other.

2. The data management system according to claim 1, wherein the one or more processors are configured to:
associate a culture condition identified by identification information read from a recording medium configured to record the identification information of the culture condition with the measurement data of the cell cultured in a vessel to which the recording medium is attached, and
control storing, in the memory, the culture condition and the associated measurement data.

3. The data management system according to claim 1, wherein
the one or more processors are configured to control storing, in the memory, a first culture condition for a first cell and a second culture condition for a second cell passaged from the first cell in association with each other such that a parent-child relationship is represented.

4. The data management system according to claim 1, wherein
the culture condition includes at least one of a condition related to the cell to be cultured, a condition related to a vessel to be used for culture, a condition related to a culture medium to be used for culture, and a condition related to a reagent to be used for culture.

5. The data management system according to claim 1, wherein the one or more processors are configured to:
selectively display one or more of the culture conditions and one or more pieces of the measurement data, and
control storing, in the memory, one or more of the culture conditions selected from one or more of the displayed culture conditions and one or more pieces of the measurement data selected from one or more pieces of the displayed measurement data in association with each other.

6. The data management system according to claim 1, wherein
a plurality of the culture conditions is associated with one or more pieces of the measurement data when stored in the memory, and
the one or more processors output one or more pieces of the measurement data associated with the plurality of the culture conditions stored in the memory in a comparable manner.

7. The data management system according to claim 1, wherein
the one or more processors are configured to classify a plurality of the culture conditions at least partially matching each other into a same group and control storing the classified plurality of the culture conditions in the memory.

8. The data management system according to claim 1, wherein
the one or more processors are configured to control storing, in the memory, job data for managing one or more jobs related to cell culture based on one or more of the culture conditions, in association with corresponding one or more culture conditions.

9. The data management system according to claim 8, wherein the one or more processors are configured to:
output one or more pieces of the measurement data associated with the one or more of the culture conditions stored in the memory, in a comparable manner.

10. A data management method implemented by a computer, the method comprising:
a first storage control step of controlling storing, in a memory, one or more culture conditions indicating conditions for culturing a cell;
a second storage control step of controlling storing, in the memory, one or more pieces of measurement data that are received via a network and that are for the cell cultured based on any one of the culture conditions; and
a third storage control step of controlling storing, in the memory, one or more pieces of the measurement data designated among the pieces of the measurement data and one or more of the culture conditions designated among the culture conditions, in association with each other.

11. A computer program product having a non-transitory computer readable medium including programmed instructions stored thereon, wherein the instructions, when executed by a computer, cause the computer to perform:
a first storage control step of controlling storing, in a memory, one or more culture conditions indicating conditions for culturing a cell;
a second storage control step of controlling storing, in the memory, one or more pieces of measurement data that are received via a network and that are for the cell cultured based on any one of the culture conditions; and
a third storage control step of controlling storing, in the memory, one or more pieces of the measurement data designated among the pieces of the measurement data and one or more of the culture conditions designated among the culture conditions, in association with each other.
